(19) United States
(12) Patent Application Publication
(10) Pub. No.: US 2013/0123341 A1
(43) Pub. Date: May 16, 2013

(54) Methods, compositions and kits for diagnosing and treating Alzheimer's disease using mitochondrial CO3 gene mutations

(71) Applicant: UNIVERSITY OF VIRGINIA PATENT FOUNDATIO, Charlottesville, VA (US)

(72) Inventor: W. Davis Parker, JR., Charlottesville, VA (US)

(73) Assignee: UNIVERSITY OF VIRGINIA PATENT FOUNDATION, Charlottesville, VA (US)

(21) Appl. No.: 13/736,298

(22) Filed: Jan. 8, 2013

Related U.S. Application Data
(63) Continuation of application No. PCT/US2011/043376, filed on Jul. 8, 2011.
(60) Provisional application No. 61/362,450, filed on Jul. 8, 2010.

(51) Int. Cl. C12Q 1/68

(52) U.S. Cl. CPC C12Q 1/6883
USPC 514/44 A; 435/6.11

(57) ABSTRACT

Methods and kits are provided for diagnosing, prognosing and treating Alzheimer's disease (AD) by identifying heteroplasmic mitochondrial mutations in cytochrome c oxidase subunit 3 (CO3). The methods are efficient, economical, and rapid, for diagnosis, prognosis and subsequent early treatment of AD in subjects.

### RELATED APPLICATION

This application claims the benefit of U.S. provisional application 61/362,450 filed Jul. 8, 2010, which is hereby incorporated by reference in its entirety.

### TECHNICAL FIELD

Compositions, methods, and kits for diagnosing and treating Alzheimer's disease, and methods for identifying a modulator of mitochondrial genes for treating Alzheimer's disease are provided herein.

### BACKGROUND

Alzheimer's disease (AD) is irreversible, progressive, and destroys memory and cognitive skills. AD is the most common cause of dementia among older people, affecting as many as 5.1 million Americans. The disease occurs in two forms: familial (FAD) and sporadic, the former following Mendelian inheritance. FAD is diagnosed in part by relevant family history and by indicia of the condition including degeneration or reduction in language, memory, perception, behavior, personality, and cognitive skill. Neither of these methods of diagnosis is quantitative and often leads to a diagnosis only after the FAD has pathologically progressed.

The second and more common type of AD, sporadic AD (SAD), is of unknown cause. SAD is not inherited according to any known Mendelian pattern, and diagnosis of SAD is accomplished by clinical findings. Diagnosis has been attempted using neuroradiologic procedures such as magnetic resonance imaging (MRI) scans and neuropsychological tests.

A quantitative method of prognosing and diagnosing both types of Alzheimer's disease, especially SAD, remains a complicated and unattained goal of medical research. There remains a need for a rapid, efficient, and universal method for determining presence of AD, a condition which has been difficult or impossible to prognose or diagnose with any commercially available method.

### SUMMARY

An aspect of the invention provides diagnosing a presence or a risk for a human subject to develop AD, the method including determining a presence in the subject of a heteroplasmic amino acid changing mutation in a region of a gene encoding a mitochondrial cytochrome c oxidase subunit III (CO3) gene, such that the region includes codons 45-250, such that the presence of the heteroplasmic amino acid changing mutation encoding one or more of codons 45-250 of the gene is an indication that the human subject has the risk to develop AD. Alternatively, the method includes determining an absence of the heteroplasmic amino acid changing mutation in the region of a gene encoding the CO3 gene, such that the absence of the mutation is an indication that the human subject is not at risk to develop AD.

In embodiments of the method, the mutation includes at least one nucleotide change in the CO3 gene selected from the group of: a substitution, a deletion, an addition. In various embodiments, the sample includes at least one of: a cell, a fluid, and a tissue. In certain embodiments of the method, the fluid is at least one selected from: blood, serum, plasma, mucus, saliva, cerebrospinal fluid, semen, tear, and urine. In certain embodiments, the cell or the tissue is selected from: vascular, epithelial, endothelial, dermal, dental, connective, muscular, neuronal, facial, cranial, soft tissue, cartilage and collagen, brain, bone, bone marrow, joint tissue, and articular joints. In certain embodiments, a control sample is obtained from an autopsy. For example, the autopsy has been determined to have had AD, for example FAD or SAD. Alternatively, the control sample is analyzed and is determined not to have any type of AD, i.e., FAD and SAD.

In various embodiments, the method involves determining the presence of absence of the mutation over a period of time, for example days, months, or years. Alternatively, the method involves comparing the mutation to a database containing a plurality of mutations.

In various embodiments of the method, the mutation is determined or identified in codon 45 to codon 60 of CO3. In various embodiments, the CO3 mutation is identified in codon 64 to codon 103, codon 75 to codon 100, codon 120 to codon 145, or codon 225 to codon 250. In various embodiments, a plurality of mutations is identified, for example in at least two regions such as codon 64 to codon 103, codon 75 to codon 100, codon 120 to codon 145, and codon 225 to codon 250.

The method in various embodiments further includes obtaining nucleic acid from the sample. In related embodiments, obtaining the nucleic acid involves at least one selected from the group of: breaking open a cell to expose the nucleic acid/nucleotide sequences within the cell, for example by cell disruption or cell lysis; disrupting or removing a membrane lipid such as by contacting a cell with a detergent; and precipitating the nucleic acid and/or a nucleotide sequence. For example precipitating includes using a precipitation agent, e.g., an alcohol, a surfactant, and a salt.

The method in various embodiments further includes amplifying at least one nucleotide sequence, such that amplifying involves hybridizing to at least one primer or probe specific for a portion of the extracted nucleic acid. In various embodiments of the method, amplifying includes at least one method and/or technique selected from: polymerase chain reaction (RT-PCR), branched DNA signal amplification, ligase chain reaction, isothermal nucleic acid sequence based amplification (NASBA), Q-beta replication, transcription-based amplification, an amplifiable RNA reporter, boomerang DNA amplification, strand displacement activation, cycling probe technology, and a sequence replication assay.

In a related embodiment of the method, the primer or probe includes at least one nucleotide sequence of CO3 gene or a portion thereof. In various embodiments of the method, the primer or the probe is attached to a matrix or scaffold, for example the matrix or scaffold includes at least one selected from the group of: a metal, a plastic, and a polymer. In a related embodiment of the method, the matrix or scaffold includes an organic material for example thiol-functionalized silica.

In various embodiments the method further includes ligating at least one nucleotide sequence to a vector such as a viral vector. For example ligating includes using an appropriate ligase and/or ligation solution. In various embodiments of the method, amplifying includes constructing a plurality of clones from the extracted nucleic acid. In related embodiments, the plurality of clones includes at least about 50 clones, at least about 100 clones, at least about 150 clones, at least about 200 clones, or at least about 300 clones. In an embodiment, the method further includes detecting the presence of the heteroplasmic amino acid changing mutation in the mitochondrial nucleotide sequence.

In various embodiments of the method, detecting includes at least one selected from: electrophoresis; an array CGH, an immunoassay; an immunological detection; fluorescence; chemiluminescence; and chromatography. In a related embodiment of the method, detecting further includes analyzing using an analytical device, for example the analytical device includes a computer. Alternatively, the analytical device includes a sequence analyzer for analyzing the presence or the absence of the mutation.

In various embodiments of the method, determining further includes identifying the presence of the heteroplasmic amino acid changing mutation in a sample from a relative selected from: a sibling, a cousin, a parent, a grandparent, and a child. For example the relative is a maternal relative. Alternatively the relative is a paternal relative.

An aspect of the present invention provides use of a sequence of a mitochondrial gene selected from the group of cytochrome c oxidase (CO) subunit 1, CO subunit 2, CO subunit 3, adenosine triphosphatase (ATPase) subunit 6, and ATPase subunit 8, for at least one of treatment, diagnosis, prognosis, genetic counseling and psychosocial management related to Alzheimer's disease (AD). In a related embodiment of the use, the AD is familial AD. Alternatively, the AD is sporadic AD.

In a related embodiment, the sequence comprises a nucleotide sequence selected from the group of: SEQ ID NO: 1, SEQ ID NO: 3, SEQ ID NO: 5, SEQ ID NO: 7, and SEQ ID NO: 9. In a related embodiment of the use, the sequence includes an amino acid sequence selected from the group of: SEQ ID NO: 2, SEQ ID NO: 4, SEQ ID NO: 6, SEQ ID NO: 8, and SEQ ID NO: 10.

In various embodiments, the use further includes comparing the mitochondrial gene to a sample and determining a presence in the sample of a heteroplasmic amino acid changing mutation in a region of the gene encoding the CO3 gene, such that the region includes codons 45-250, such that the presence of the heteroplasmic amino acid changing mutation encoding one or more of codons 45-250 of the gene is an indication that the sample has a risk of developing Alzheimer's Disease.

In various embodiments, the use further includes comparing the mitochondrial gene to a sample and determining an absence in the sample of the heteroplasmic amino acid changing mutation in a region of the gene encoding the CO3 gene, such that the region includes codons 45-250, such that the absence of the heteroplasmic amino acid changing mutation encoding one or more of codons 45-250 of the gene is an indication that the sample has no risk of developing Alzheimer's Disease.

In various embodiments, the mutation includes at least one nucleotide change selected from a substitution.

In a related embodiment of the use, the sample includes at least one of: a cell, a fluid, and a tissue. In various embodiments, the fluid is at least one selected from the group of: blood, serum, plasma, mucus, saliva, cerebrospinal fluid, semen, tear, and urine. In certain embodiments of the use, the cell or the tissue is selected from at least one selected from: vascular, epithelial, endothelial, dermal, dental, connective, muscular, neuronal, facial, cranial, soft tissue, cartilage and collagen, brain, bone, and bone marrow.

### BRIEF DESCRIPTION OF THE DRAWINGS

FIG. 1 panel A is a graph of showing mutations in the gene encoding cytochrome oxidase 3 (CO3) as a function of codon (abscissa) of blood samples and brain samples from subjects (ordinate) diagnosed with AD or control normal subjects. The samples included: control brain samples from normal autopic subjects (samples 1-9; green circles); brain autopic samples from subjects having been diagnosed with AD (samples 10-18; red circles); blood samples from living subjects having AD (samples 19-21, 23-24 and 26-33; blue diamonds); and control blood samples from normal living subjects (samples 22, 25 and 34-36; black diamonds). The horizontal lines on the graph identify each sample. The vertical axis lines identify the CO3 codons. The dotted verticals lines represent the codon region from codon 64 to codon 103. Data show low abundance, amino acid changing mutations at specific codons.

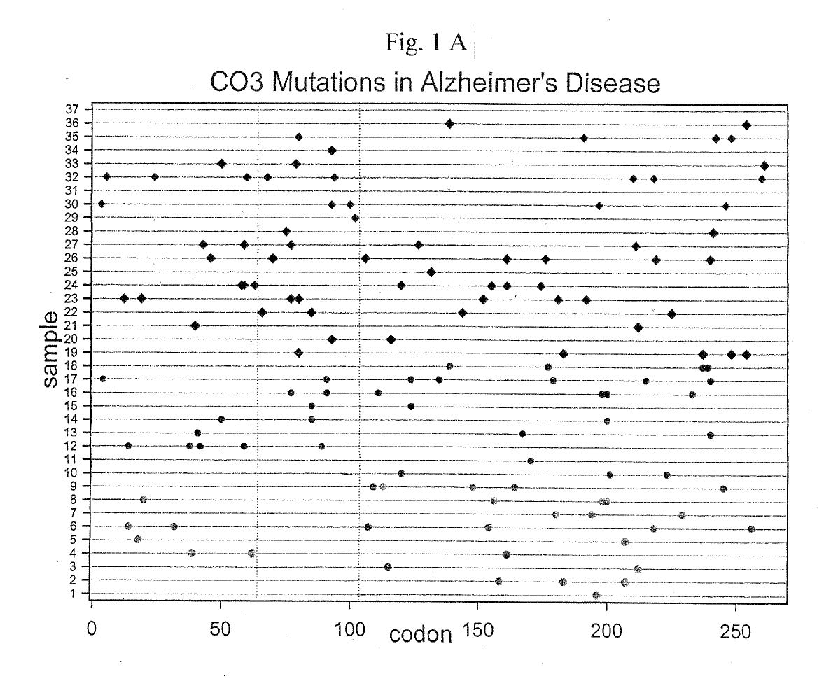

FIG. 1 panel B is a graph of showing mutations in the gene encoding cytochrome oxidase 3 (CO3) as a function of codon number (abscissa) of brain samples from subjects (ordinate) diagnosed with AD or control normal subjects. The samples included control brain samples from normal autopic subjects (circles) and brain autopic samples from subjects having been diagnosed with AD (squares). Brain sample data in FIG. 1 panel B is the same data as the brain data in FIG. 1 panel A.

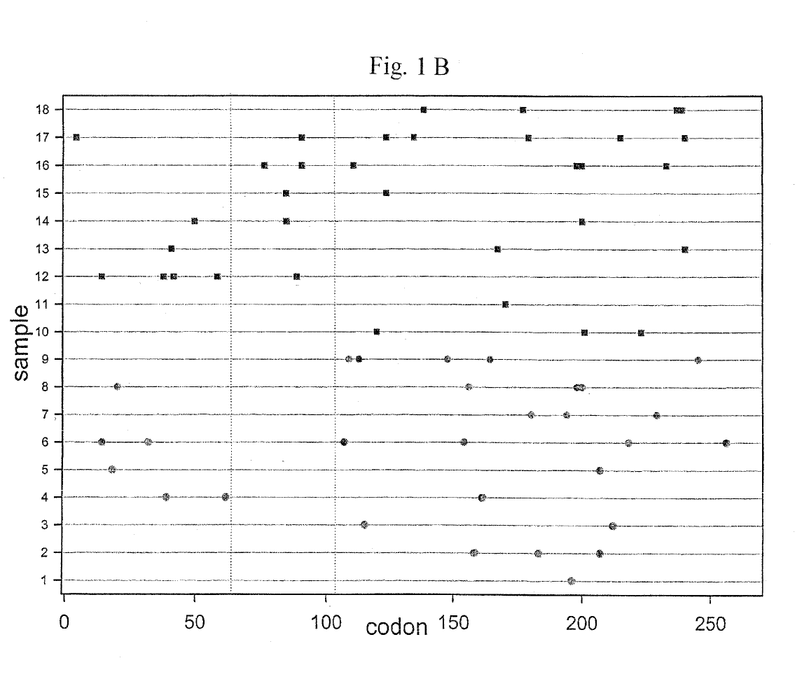

FIG. 1 panel C is a graph of showing mutations in the gene encoding cytochrome oxidase 3 (CO3) as a function of codon (abscissa) of blood samples from subjects (ordinate) diagnosed with AD or control normal subjects. The samples included control blood samples from normal living subjects (stars) and blood samples from living subjects having been diagnosed with AD (diamonds). Blood data in FIG. 1 panel C is the same data as the blood data in FIG. 1 panel A.

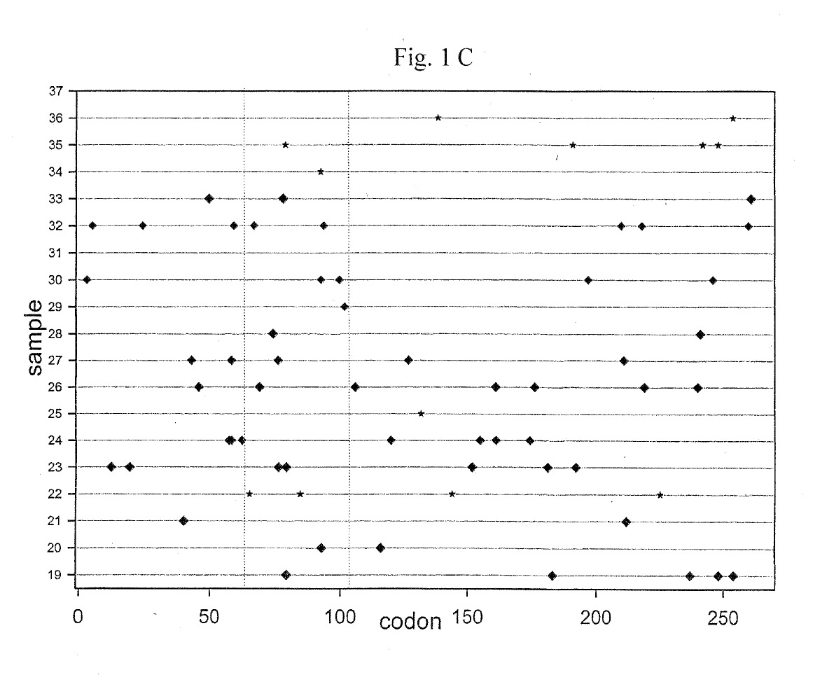

### DETAILED DESCRIPTION

### Mitochondrial Mutations and Diagnosis and Prognosis of AD

Mitochondria are self-replicating organelles that occur in varying numbers, shapes, and sizes in the cytoplasm of eukaryotic cells, and are the site of tissue respiration. The number of mitochondria per cell depends on the cells and specifically the energy needs of the cells. For example, the number of mitochondria in a cell varies from about 1000 to about 10,000, for example the average human brain contains about 100 billions cells each having about 1,000 to 2,000 mitochondria per cell. See Dimauro et al. 2005 Ann Med. 37(3):222-32.

Further each mitochondrion contains DNA including a number of mitochondrial genes, for example about two to ten mitochondrial DNA copies, each encoding 37 genes. See Finsterer et al. 2009 Eur J Neurol. 16(12):1255-1264 and Kang et al. 2005 Curr Med Chem 12(4):429-41. Cytochrome c oxidase (CO or COX) is a terminal component of the electron transport chain located in the mitochondria of eukaryotic cells. Cytochrome c oxidase, also referred to as complex IV of the electron transport chain, is composed of at least thirteen subunits. Ten of these CO subunits are encoded by nuclear genes; three other subunits (CO1, CO2, and CO3) are encoded by mitochondrial genes. Mitochondrial DNA (mtDNA) is a small circular DNA molecule that is approximately 17 kiloases (kB) in length in humans. The mtDNA encodes for two ribosomal RNAs (rRNA), a complete set of transfer RNAs (tRNA) and thirteen proteins, including three cytochrome c oxidase subunits COX I, COX II, and COX III (Herrnstadt et al. U.S. Pat. No. 6,171,859 issued Jan. 9, 2001). The catalytic centers of CO are encoded by two mitochondrial genes, CO1 and CO2 (encoding CO subunits I and II, respectively).

The mtDNA present in an individual is inherited primarily from mtDNA in the ovum at the time of conception. Mutations in a nucleotide sequence mtDNA found in all copies of mtDNA in an individual are referred to as “homoplasmic”. Mutations that affect only a portion of the copies of mtDNA are referred to as “heteroplasmic”, and proportions of mutant and wildtype vary among different mitochondria in the same individual. The majority of mitochondrially encoded proteins and all mitochondrially encoded COX proteins are transcribed from the heavy strand of mtDNA. The “heavy” and “light” strands are separated on the basis of density. Park et al. 2011 J. Cell Biol. 193(5): 809-818.

Lu et al. 2010 J Alzheimers Dis. 21(1): 141-154 shows that cytochrome c oxidase (CO) is kinetically abnormal, and activity of the enzyme is decreased in brain and peripheral tissue in late-onset AD. CO is encoded by both the mitochondrial and the nuclear genomes.

Examples herein analyzed nucleotide sequences of five different genes, including cytochrome c oxidase genes CO1, CO2, and CO3, adenosine triphosphatase (ATPase) 6 gene, and ATPase-8 gene in subjects, to determine presence of heteroplasmic mutations that alter CO activity and correlate with AD. In the Examples herein, specific heteroplasmic mutations in the mtDNA CO3 gene were identified and shown to segregate at a higher frequency with AD subjects compared with normal subjects in both brain samples and blood samples. These mutations were identified in specific codon regions of the mtDNA CO3 gene and show that specific mutant CO3 mitochondrial genes result in AD conditions. As mitochondrial mutations are maternally inherited, these mutations are of value in treatment, diagnosis, prognosis, genetic counseling and psychosocial management.

Examples herein have identified a set of gene mutations that correlate with the presence of SAD. Detailed analysis of the nucleotide sequences of mitochondrial genes CO1 (SEQ ID NO:1), CO2 (SEQ ID NO:3), CO3 (SEQ ID NO:5), ATPase 6 (SEQ ID NO:7), and ATPase 8 (SEQ ID NO:9) recovered from the tissue of AD patients indicate that presence of low abundance, amino acid changing mutations in at least one region of the CO3 gene correlates highly with the diagnosis of AD. These CO3 gene mutations are of pathological significance because of their specific correlation with prognosis or diagnosis of AD in subjects. Identification and determination of these gene mutations in various cells and tissues taken from patients are useful in diagnosing FAD and SAD.

It is envisioned that the methods and kits herein are useful to diagnose at risk populations, particularly those individuals that do not have a clear family history. Thus, the present invention includes compositions and methods useful for diagnosing a disease or disorder associated with the mutations disclosed herein. Examples herein further encompass the use of combinations of any of the sets of codon or protein biomarkers or combinations thereof, described herein for use in diagnosing, predicting the prognosis, predicting the onset or development, or monitoring the progression of AD.

A presence of low abundance, heteroplasmic, amino acid changing mutations in CO3 codons, for example codons 64 through 103 of the mitochondrial CO3 gene, is diagnostic for AD in most patients and is useful for diagnosing or prognosing SAD and FAD.

The present invention encompasses diagnosing the conditions described by analyzing samples from target subjects. Samples include, but are not limited to, tissue samples including archival samples, biopsies, serum, blood, plasma, saliva, feces, cerebrospinal fluid, semen, tears, and urine, and other samples obtained from a subject of interest.

The present invention further encompasses compositions and methods useful for suppressing or selectively eliminating these mutations in a course of treatment of AD. The present invention provides therapeutic agents for treating a subject having AD or preventing the pathological symptoms of AD including contacting a subject with a therapeutic agent that negatively modulates at least one amino acid changing mutation in a small region of the CO3 gene. For example contacting the subject comprises contacting with a virus vector that expresses the therapeutic agent including a negative modulator of expression of the mutation.

The present invention provides a method of treating a human subject having AD or at risk for AD, the method including: identifying at least one mismatch in a CO3 gene between a human control subject not having AD and the subject, such that the mismatch includes at least one heteroplasmic mismatch or a plurality of heteroplasmic mismatches; determining a nucleic acid sequence encoding a region having the mismatch or among domains having the plurality of mismatches, and constructing a negative modulator of the nucleic acid encoding the region having the mismatch or the regions having the plurality of mismatches; and contacting at least one of cells or tissue of the subject with the vector that expresses the modulator and down regulates or eliminates expression of the region or regions, thereby treating the subject.

The methods herein include engineering the modulator by constructing a small interfering RNA (siRNA) that specifically targets a nucleic acid encoding the mismatch or mismatches or encoding an agent that binds to the mismatch or mismatches.

The modulator in various embodiments of the method includes a nucleic acid vector or a virus vector that includes a nucleic acid sequence encoding a siRNA negative modulator of a target nucleic acid encoding the mismatch or mismatches, such that the siRNA down-regulates or interferes with the function of mRNAs encoding the mismatch or mismatches. In certain embodiments, the virus vector includes a nucleic acid sequence encoding an antisense RNA modulator of a target nucleic acid, for example the target nucleic acid includes conserved domains and polymorphic domains. Alternatively, the vector is a bacterial vector.

Methods for constructing synthetic siRNA or an antisense expression cassette and inserting it into a recombinantly engineered nucleic acid of a vector are well known in the art and are shown for example in Reich et al. U.S. Pat. No. 7,847,090 issued Dec. 7, 2010; Reich et al. U.S. Pat. No. 7,674,895 issued Mar. 9, 2010; and Khvorova et al. U.S. Pat. No. 7,642,349 issued Jan. 5, 2010, each of which is incorporated herein in its entirety. For example, the invention herein includes synthetic siRNAs that include a sense RNA strand and an antisense RNA strand, such that the sense RNA strand includes a nucleotide sequence substantially identical to a target nucleic acid sequence in cells. Thus, under the circumstances of cells being contacted with viral vectors encoding the siRNAs, the cells express the siRNAs that then negatively modulate expression of the target nucleic acid sequence, e.g., a target nucleotide sequence in a CO3 gene. Methods and compositions for targeting and treating cytochrome c oxidase dysfunction are shown also in Davis et al. U.S. Pat. No. 6,867,197 issued Mar. 15, 2005, each of which is incorporated herein in its entirety. In certain embodiments, the modulator, for example the siRNA, is permanently integrated into a genome of the cells or tissue of the subject.

Methods herein include contacting the tissue selected from: brain, cerebrospinal fluid, ganglia, kidney, pancreas, liver, heart, lung, stem cells, cornea, bone marrow, fetal cord blood, and peripheral blood mononuclear cells. In certain embodiments of the method, the stem cells are selected from brain stem cells, hematopoietic stem cells, hemangioblast cells, mesenchymal stem cells, hepatocyte stem cells, pancreatic stem cells, pulmonary stem cells, neural stem cells, fetal stem cells, and embryonic stem cells. In certain embodiments, the method including contacting the tissue which is an organ, for example selected from the group of: brain; kidney; pancreas; liver; heart; lung; spinal cord; and cornea. Alternatively, the tissue is bone marrow.

The methods in related embodiments further involve preparing a nucleotide sequence encoding a modulator that binds to the mismatch or mismatches, or that encodes a recombinant CO3 enzyme having no mutations.

Methods herein include a modulator that comprises a recombinant normal version of cytochrome c oxidase enzyme. Methods of making recombinant cytochrome c oxidase enzyme complexes are shown in Asakura et al. U.S. Pat. No. 7,842,483 issued Nov. 30, 2010; and Asakura et al. U.S. Pat. No. 6,869,785 issued Mar. 22, 2005, each of which is incorporated herein in its entirety.

The present invention provides a kit for use in treating a human subject having AD or at risk for AD, the kit including: a modulator that negatively modulates expression of a target nucleic acid sequence encoding at least one heteroplasmic mismatch or a plurality of heteroplasmic mismatches in a CO3 gene, instructions for use, and a container. In certain embodiments of the kit, the modulator includes a small interfering RNA (siRNA) that specifically targets a nucleic acid encoding the mismatch or mismatches or encoding an agent that binds to the mismatch or mismatches. For example the siRNA targets a CO3 gene mutation that is identified in codon 64 to codon 103, codon 75 to codon 100, codon 120 to codon 145, or codon 225 to codon 250.

The present invention provides a product including a modulator that negatively modulates expression of a target nucleic acid sequence encoding at least one heteroplasmic mismatch or a plurality of heteroplasmic mismatches in a CO3 gene, and an appropriate carrier; such that the modulator is for use in treating a human subject having AD or at risk for AD. In various embodiments, the product is compounded or formulated in an effective dose for treating the subject. An aspect of the invention provides use of the product in the preparation of a medicament for treating a human subject having AD or at risk for AD.

### DEFINITIONS

In describing and claiming the invention, the following terminology will be used in accordance with the definitions set forth below. Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described herein. As used herein, each of the following terms has the meaning associated with it in this section. Specific and preferred values listed below for radicals, substituents, and ranges are for illustration only; they do not exclude other defined values or other values within defined ranges for the radicals and substituents.

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about”.

The terms “additional therapeutically active compound” or “additional therapeutic agent”, as used in the context of the present invention, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated.

As use herein, the terms “administration of” and or “administering” a compound should be understood to mean providing a compound of the invention or a prodrug of a compound of the invention to a subject in need of treatment.

The term “adult” as used herein, is meant to refer to any non-embryonic or non-juvenile subject. For example the term “adult adipose tissue stem cell,” refers to an adipose stem cell, other than that obtained from an embryo or juvenile subject.

Cells or tissue are “affected” by an injury, disease or disorder if the cells or tissue have an altered phenotype relative to the same cells or tissue in a subject not afflicted with the injury, disease, condition, or disorder.

As used herein, an “agonist” is a composition of matter that, when administered to a mammal such as a human, enhances or extends a biological activity of interest. Such effect may be direct or indirect.

A disease, condition, or disorder is “alleviated” if the severity of a symptom of the disease or disorder, the frequency with which such a symptom is experienced by a patient, or both, are reduced.

As used herein, “alleviating an injury, disease or disorder symptom,” means reducing the frequency or severity of the symptom.

As used herein, amino acids are represented by the full name thereof, by the three letter code corresponding thereto, or by the one-letter code corresponding thereto, as indicated in the following table:

| Full Name | Three-Letter Code | One-Letter Code |
|---|---|---|
| Aspartic Acid | Asp | D |
| Glutamic Acid | Glu | E |
| Lysine | Lys | K |
| Arginine | Arg | R |
| Histidine | His | H |
| Tyrosine | Tyr | Y |
| Cysteine | Cys | C |
| Asparagine | Asn | N |
| Glutamine | Gln | Q |
| Serine | Ser | S |
| Threonine | Thr | T |
| Glycine | Gly | G |
| Alanine | Ala | A |
| Valine | Val | V |
| Leucine | Leu | L |
| Isoleucine | Ile | I |
| Methionine | Met | M |
| Proline | Pro | P |
| Phenylalanine | Phe | F |
| Tryptophan | Trp | W |

The term “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the present invention, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the invention.

The term “amino acid” is used interchangeably with “amino acid residue,” and may refer to a free amino acid and to an amino acid residue of a peptide. It will be apparent from the context in which the term is used whether it refers to a free amino acid or a residue of a peptide.

Amino acids have the following general structure:

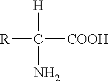

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

The nomenclature used to describe the peptide compounds of the present invention follows the conventional practice wherein the amino group is presented to the left and the carboxy group to the right of each amino acid residue. In the formulae representing selected specific embodiments of the present invention, the amino- and carboxy-terminal groups, although not specifically shown, will be understood to be in the form they would assume at physiologic pH values, unless otherwise specified.

The term “basic” or “positively charged” amino acid as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter that when administered to a mammal such as a human, inhibits or impedes a biological activity attributable to the level or presence of an endogenous compound in the mammal. Such effect may be direct or indirect.

The term “antimicrobial agents” as used herein refers to any naturally-occurring, synthetic, or semi-synthetic compound or composition or mixture thereof, which is safe for human or animal use as practiced in the methods of this invention, and is effective in killing or substantially inhibiting the growth of microbes. “Antimicrobial” as used herein, includes antibacterial, antifungal, and antiviral agents.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, Fv, Fab and F(ab)₂, as well as single chain antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426).

An “antibody heavy chain,” as used herein, refers to the larger of the two types of polypeptide chains present in all antibody molecules.

An “antibody light chain,” as used herein, refers to the smaller of the two types of polypeptide chains present in all antibody molecules.

By the term “synthetic antibody” as used herein, is meant an antibody which is generated using recombinant DNA technology, such as, for example, an antibody expressed by a bacteriophage as described herein. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using synthetic DNA or amino acid sequence technology which is available and well known in the art.

The term “antigen” as used herein is defined as a molecule that provokes an immune response. This immune response may involve either antibody production, or the activation of specific immunologically-competent cells, or both. An antigen can be derived from organisms, subunits of proteins/antigens, killed or inactivated whole cells or lysates.

A ligand or a receptor (e.g., an antibody) “specifically binds to” or “is specifically immunoreactive with” a compound when the ligand or receptor functions in a binding reaction which is determinative of the presence of the compound in a sample of heterogeneous compounds. Thus, under designated assay (e.g., immunoassay) conditions, the ligand or receptor binds preferentially to a particular compound and does not bind in a significant amount to other compounds present in the sample. For example, a polynucleotide specifically binds under hybridization conditions to a compound polynucleotide comprising a complementary sequence; an antibody specifically binds under immunoassay conditions to an antigen bearing an epitope against which the antibody was raised. A variety of immunoassay formats may be used to select antibodies specifically immunoreactive with a particular protein. For example, solid-phase ELISA immunoassays are routinely used to select monoclonal antibodies specifically immunoreactive with a protein. See Harlow and Lane (1988, Antibodies, A Laboratory Manual, Cold Spring Harbor Publications, New York) for a description of immunoassay formats and conditions that can be used to determine specific immunoreactivity.

The term “antigenic determinant” as used herein refers to that portion of an antigen that makes contact with a particular antibody (i.e., an epitope). When a protein or fragment of a protein, or chemical moiety is used to immunize a host animal, numerous regions of the antigen may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

As used herein, the term “antisense oligonucleotide” or antisense nucleic acid means a nucleic acid polymer, at least a portion of which is complementary to a nucleic acid which is present in a normal cell or in an affected cell. “Antisense” refers particularly to the nucleic acid sequence of the non-coding strand of a double stranded DNA molecule encoding a protein, or to a sequence which is substantially homologous to the non-coding strand. As defined herein, an antisense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the invention include, but are not limited to, phosphorothioate oligonucleotides and other modifications of oligonucleotides.

The term “biological sample,” as used herein, refers to samples obtained from a living organism, including skin, hair, tissue, blood, plasma, cells, sweat, and urine.

As used herein, the term “biologically active fragments” or “bioactive fragment” of the polypeptides encompasses natural or synthetic portions of the full-length protein that are capable of specific binding to their natural ligand or of performing the function of the protein.

A “biomarker” is a specific biochemical in the body which has a particular molecular feature that makes it useful for measuring the progress of disease or the effects of treatment, or for measuring a process of interest.

“Cancer” or “malignancy” are used as synonymous terms and refer to any of a number of diseases that are characterized by uncontrolled, abnormal proliferation of cells, the ability of affected cells to spread locally or through the bloodstream and lymphatic system to other parts of the body (i.e., metastasize), as well as any of a number of characteristic structural and/or molecular features. A “cancerous” or “malignant cell” is understood as a cell having specific structural properties, lacking differentiation and being capable of invasion and metastasis. Examples of cancers are, breast, lung, brain, bone, liver, kidney, colon, and prostate cancer. (see DeVita, V. et al. (eds.), 2001, Cancer Principles and Practice of Oncology, 6th. Ed., Lippincott Williams & Wilkins, Philadelphia, Pa.; this reference is herein incorporated by reference in its entirety for all purposes).

“Cancer-associated” refers to the relationship of a nucleic acid and its expression, or lack thereof, or a protein and its level or activity, or lack thereof, to the onset of malignancy in a subject cell. For example, cancer can be associated with expression of a particular gene that is not expressed, or is expressed at a lower level, in a normal healthy cell. Conversely, a cancer-associated gene can be one that is not expressed in a malignant cell (or in a cell undergoing transformation), or is expressed at a lower level in the malignant cell than it is expressed in a normal healthy cell.

As used herein, the term “carrier molecule” refers to any molecule that is chemically conjugated to the antigen of interest that enables an immune response resulting in antibodies specific to the native antigen.

As used herein, the term “chemically conjugated,” or “conjugating chemically” refers to linking the antigen to the carrier molecule. This linking can occur on the genetic level using recombinant technology, wherein a hybrid protein may be produced containing the amino acid sequences, or portions thereof, of both the antigen and the carrier molecule. This hybrid protein is produced by an oligonucleotide sequence encoding both the antigen and the carrier molecule, or portions thereof. This linking also includes covalent bonds created between the antigen and the carrier protein using other chemical reactions, such as, but not limited to glutaraldehyde reactions. Covalent bonds may also be created using a third molecule bridging the antigen to the carrier molecule. These cross-linkers are able to react with groups, such as but not limited to, primary amines, sulfhydryls, carbonyls, carbohydrates or carboxylic acids, on the antigen and the carrier molecule. Chemical conjugation also includes non-covalent linkage between the antigen and the carrier molecule.

The term “competitive sequence” refers to a peptide or a modification, fragment, derivative, or homolog thereof that competes with another peptide for its cognate binding site.

“Complementary” refers to the broad concept of sequence complementarity between regions of two nucleic acid strands or between two regions of the same nucleic acid strand. It is known that an adenine residue of a first nucleic acid region is capable of forming specific hydrogen bonds (“base pairing”) with a residue of a second nucleic acid region which is antiparallel to the first region if the residue is thymine or uracil. As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.”

Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. Preferably, the first region comprises a first portion and the second region comprises a second portion, whereby, when the first and second portions are arranged in an antiparallel fashion, at least about 50%, and preferably at least about 75%, at least about 90%, or at least about 95% of the nucleotide residues of the first portion are capable of base pairing with nucleotide residues in the second portion. More preferably, all nucleotide residues of the first portion are capable of base pairing with nucleotide residues in the second portion.

The term “complex”, as used herein in reference to proteins, refers to binding or interaction of two or more proteins. Complex formation or interaction can include such things as binding, changes in tertiary structure, and modification of one protein by another, such as phosphorylation.

A “compound,” as used herein, refers to any type of substance or agent that is commonly considered a drug, or a candidate for use as a drug, as well as combinations and mixtures of the above.

As used herein, the term “conservative amino acid substitution” is defined herein as an amino acid exchange within one of the following five groups:

I. Small aliphatic, nonpolar or slightly polar residues:
Ala, Ser, Thr, Pro, Gly;

II. Polar, negatively charged residues and their amides:
Asp, Asn, Glu, Gln;

III. Polar, positively charged residues:
His, Arg, Lys;

IV. Large, aliphatic, nonpolar residues:
Met Leu, Ile, Val, Cys

V. Large, aromatic residues:
Phe, Tyr, Trp

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, the term “diagnosis” refers to detecting cancer or a risk or propensity for development of cancer, for the types of cancer encompassed by the invention. In any method of diagnosis there exist false positives and false negatives. Any one method of diagnosis does not provide 100% accuracy.

A “disease” is a state of health of an animal wherein the subject cannot maintain homeostasis, and wherein if the disease is not ameliorated then the subject's health continues to deteriorate. In contrast, a “disorder” in an subject is a state of health in which the animal is able to maintain homeostasis, but in which the subject's state of health is less favorable than it would be in the absence of the disorder. Left untreated, a disorder does not necessarily cause a further decrease in the subject's state of health.

As used herein, an “effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

An “enhancer” is a DNA regulatory element that can increase the efficiency of transcription, regardless of the distance or orientation of the enhancer relative to the start site of transcription.

The term “epitope” as used herein is defined as small chemical groups on the antigen molecule that can elicit and react with an antibody. An antigen can have one or more epitopes. Most antigens have many epitopes; i.e., they are multivalent. In general, an epitope is roughly 5 amino acids or sugars in size. One skilled in the art understands that generally the overall three-dimensional structure, rather than the specific linear sequence of the molecule, is the main criterion of antigenic specificity.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” molecule is a molecule in a form in which it exhibits a property or activity by which it is characterized. A functional enzyme, for example, is one that exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology.

As used herein, “homology” is used synonymously with “identity.”

The determination of percent identity between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

The term “inhibit,” as used herein, refers to the ability of a compound, agent, or method to reduce or impede a described function, level, activity, rate, etc., based on the context in which the term “inhibit” is used. Preferably, inhibition is by at least 10%, more preferably by at least 25%, even more preferably by at least 50%, and most preferably, the function is inhibited by at least 75%. The term “inhibit” is used interchangeably with “reduce” and “block.”

As used herein “injecting or applying” includes administration of a compound of the invention by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

An “isolated nucleic acid” refers to a nucleic acid segment or fragment which has been separated from sequences which flank it in a naturally occurring state, e.g., a DNA fragment which has been removed from the sequences which are normally adjacent to the fragment, e.g., the sequences adjacent to the fragment in a genome in which it naturally occurs. The term also applies to nucleic acids which have been substantially purified from other components which naturally accompany the nucleic acid, e.g., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA which is part of a hybrid gene encoding additional polypeptide sequence.

As used herein the term “expression” when used in reference to a gene or protein, without further modification, is intended to encompass transcription of a gene and/or translation of the transcript into a protein.

“Malexpression” of a gene means expression of a gene in a cell of a patient afflicted with a disease or disorder, wherein the level of expression (including non-expression), the portion of the gene expressed, or the timing of the expression of the gene with regard to the cell cycle, differs from expression of the same gene in a cell of a patient not afflicted with the disease or disorder. It is understood that malexpression may cause or contribute to the disease or disorder, be a symptom of the disease or disorder, or both.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions.

The term “material” refers to any compound, molecule, substance, or group or combination thereof that forms any type of structure or group of structures during or after electroprocessing. Materials include natural materials, synthetic materials, or combinations thereof. Naturally occurring organic materials include any substances naturally found in the body of plants or other organisms, regardless of whether those materials have or can be produced or altered synthetically. Synthetic materials include any materials prepared through any method of artificial synthesis, processing, or manufacture. Preferably, the materials are biologically compatible materials.

As used herein, the terms “native”, “natural” “native antigen”, or “natural antigen” refers to the antigen as it occurs in nature. With respect to the invention, the “native antigens” are of “low immunogenicity”. “Low immunogenicity” refers to the inability of the natural molecule to elicit a strong immune response resulting in the production of high affinity antibodies. The term “antigen”, “antigen of interest,” or specific molecules, such as the cancer-testis antigens encompassed herein include the whole molecule or any portions thereof that maintain antigenic distinctiveness specific for the native antigen.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

The term “Oligonucleotide” typically refers to short polynucleotides, generally no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

“Operably linked” refers to a juxtaposition wherein the components are configured so as to perform their usual function. Thus, control sequences or promoters operably linked to a coding sequence are capable of effecting the expression of the coding sequence. By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

By “presensitization” is meant pre-administration of at least one innate immune system stimulator prior to challenge with a pathogenic agent. This is sometimes referred to as induction of tolerance.

A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

The term “prevent,” as used herein, means to stop something from happening, or taking advance measures against something possible or probable from happening. In the context of medicine, “prevention” generally refers to action taken to decrease the chance of getting a disease or condition.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

As used herein, the term “pharmaceutically acceptable carrier” includes any of the standard pharmaceutical carriers, such as a phosphate buffered saline solution, water, emulsions such as an oil/water or water/oil emulsion, and various types of wetting agents. The term also encompasses any of the agents approved by a regulatory agency of the US Federal government or listed in the US Pharmacopeia for use in animals, including humans

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates. The terms “protein pathway” and “protein regulatory pathway” are used interchangeably herein.

The term “regulate” refers to either stimulating or inhibiting a function or activity of interest.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

As used herein, the term “secondary antibody” refers to an antibody that binds to the constant region of another antibody (the primary antibody).

By “small interfering RNAs (siRNAs)” is meant, inter alia, an isolated dsRNA molecule comprised of both a sense and an anti-sense strand. In one aspect, it is greater than 10 nucleotides in length. The term “siRNA” also refers to a single transcript which has both the sense and complementary antisense sequences from the target gene, e.g., a hairpin. SiRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

As used herein, the term “solid support” relates to a solvent insoluble substrate that is capable of forming linkages (preferably covalent bonds) with various compounds. The support can be either biological in nature, such as, without limitation, a cell or bacteriophage particle, or synthetic, such as, without limitation, an acrylamide derivative, agarose, cellulose, nylon, silica, or magnetized particles.

“Sperm-specific”, as used herein, refers to an antigen which is present at higher levels on sperm than other cells, or is exclusively present in sperm.

A “test sample”, as used herein, refers to a sample of semen or to a sample obtained as a forensic sample such as a post-coital swab.

Used interchangeably herein are the following pairs of words (1) “detect” and “identify”; (2) “select” and “isolate”; and (3) “sperm surface” and “sperm plasma membrane.”

The term “standard,” as used herein, refers to something used for comparison. For example, a standard can be a known standard agent or compound which is administered or added to a control sample and used for comparing results when measuring said compound in a test sample. In one aspect, the standard compound is added or prepared at an amount or concentration that is equivalent to a normal value for that compound in a normal subject. Standard can also refer to an “internal standard,” such as an agent or compound which is added at known amounts to a sample and is useful in determining such things as purification or recovery rates when a sample is processed or subjected to purification or extraction procedures before a marker of interest is measured.

A “subject” of analysis, diagnosis, or treatment is an animal Such animals include mammals, preferably a human.

As used herein, a “subject in need thereof” is a patient, animal, mammal, or human, who will benefit from the method of this invention.

The term “substantially pure” describes a compound, e.g., a protein or polypeptide which has been separated from components which naturally accompany it. Typically, a compound is substantially pure when at least 10%, more preferably at least 20%, more preferably at least 50%, more preferably at least 60%, more preferably at least 75%, more preferably at least 90%, and most preferably at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cells united to perform a specific function; (2) a part of an organism consisting of an aggregate of cells having a similar structure and function; or (3) a grouping of cells that are similarly characterized by their structure and function, such as muscle or nerve tissue.

The term to “treat,” as used herein, means reducing the frequency with which symptoms are experienced by a patient or subject or administering an agent or compound to reduce the frequency with which symptoms are experienced.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease.

“Treating” is used interchangeably with “treatment” herein.

By the term “vaccine,” as used herein, is meant a composition which when inoculated into an animal has the effect of stimulating an immune response in the subject, which serves to fully or partially protect the subject against a disease or its symptoms. The term vaccine encompasses prophylactic as well as therapeutic vaccines. A combination vaccine is one which combines two or more vaccines, or two or more compounds or agents.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

### EXAMPLES

### Example 1

### DNA Extraction from Blood Cells and Brain Tissues

Nine brain samples from subjects diagnosed with AD and nine control brain samples from normal control subjects were obtained from the Brain Research Facility brainbank at the University of Virginia in Charlottesville, Va. Each brain sample was obtained from the frontal cortex of autopsied tissue. Equivalent pieces of frontal cortex were obtained for each subject. The diagnoses of AD or normal for of each subject were verified by medical history and clinical autopsy analysis. Control brain samples were observed to have no clinical indicia of AD or any other neurodegenerative disorder.

Blood samples were collected from thirteen living subjects that were diagnosed with AD and from five living normal control subjects. Samples were collected with approval of the Institutional Review Board and with the consent of the subjects. Fresh blood samples were placed in vacuum containers containing ethylenediaminetetraacetic acid (EDTA), and were stored at 4° C. until use. The platelet-enriched white blood cell fraction was isolated from the containers within 34 hours of collection, and was centrifuged (14,000×g) for ten minutes. The resulting platelet-enriched pellet was re-suspended in 0.4 mL of 0.9% sodium chloride containing 1 mM EDTA.

Total genomic DNA was purified from the autopsy samples and blood samples using the QiaAmp kit (Qiagen Inc.; Valencia, Calif.). The concentration and purity of DNA extracts were analyzed spectrophotometrically at 260 nm. The individual extracted genomic DNA from each sample was then stored at −80° C.

Methods for extracting mitochondrial DNA (mtDNA) from blood cells and brain tissues were used as described in Parker U.S. Pat. No. 7,537,893 issued May 26, 2009, which is hereby incorporated by reference in its entirety. Additional methods and details are shown in Smigrodzki et al. Neurobiology of Aging 25 (2004) 1273, and Parker et al. 2005 Biochem. Biophys. Res. Comm. 326: 667-669, each of which is incorporated herein in its entirety.

### Example 2

### PCR Amplification

Mitochondrial DNA gene nucleotide sequences were amplified with HotStart Pfu DNA polymerase (Stratagene; Santa Clara, Calif.) following manufacturer's instructions for enzyme, dNTP and primer concentrations. The mtDNA genes sequenced were cytochrome c oxidase subunit I gene (CO1; SEQ ID NO: 1), cytochrome c oxidase subunit II gene (CO2; SEQ ID NO: 3) cytochrome c oxidase subunit III gene (CO3; SEQ ID NO:5), mitochondrially encoded ATP synthase 6 (ATP-6; SEQ ID NO: 7) and mitochondrially encoded ATP synthase 8 (ATP-8; SEQ ID NO: 9). The corresponding amino acid sequences for CO1, CO2, CO3, ATP-6 and ATP-8 are shown in SEQ ID NO: 2, SEQ ID NO: 4, SEQ ID NO: 6, SEQ ID NO: 8, and SEQ ID NO: 10 respectively.

AD tissues were shown to be associated with a loss of biochemical activity of cytochrome c oxidase, and cytoplasmic hybrids, cybrids indicated loss of cytochrome c oxidase activity in AD may be encoded by mtDNA. Lu et al. 2010 J Alzheimers Dis. 21(1): 141-154; and Swerdlow 2011 ARS AD Review 1-83, each of which is incorporated herein in its entirety. Further, ATPase mutations could secondarily depress cytochrome c oxidase activity.

TABLE 1
PCR primers in mtDNA amplification

| Name | Nucleotide sequence |
|---|---|
| SEQ ID NO.: 11 | AGCACCCTAATCAACTGGCTTCAA |
| SEQ ID NO.: 12 | CTTCGCAGGCGGCAAAGACTA |
| SEQ ID NO.: 13 | AGCACCCTAATCAACTGGCTTCAA |
| SEQ ID NO.: 14 | CTTCGCAGGCGGCAAAGACTA |
| SEQ ID NO.: 15 | AGCACCCTAATCAACTGGCTTCAA |
| SEQ ID NO.: 16 | CTTCGCAGGCGGCAAAGACTA |
| SEQ ID NO.: 17 | GCCCTAGCCCACTTCTTACC |
| SEQ ID NO.: 18 | TTAAGGCGACAGCGATTTCT |
| SEQ ID NO.: 19 | CCGACTAATCACCACCCAAC |
| SEQ ID NO.: 20 | GATGGCCATGGCTAGGTTTA |
| SEQ ID NO.: 21 | GTATGGCCCACCATAATTGC |
| SEQ ID NO.: 22 | GCAATGAATGAAGCGAACAG |

Primer pairs (SEQ ID NOs: 11-22) used in Examples herein are shown in Table 1. For PCR amplification reactions were incubated two minutes at 94° C., followed by 25 cycles of denaturation at 94° C. for 15 seconds, annealing at 55.5° C. for 30 seconds, extension at 72° C. for one minute, and a final extension step at 72° C. for seven minutes. The amplifications were performed in 100 microliter (μl) volumes using 100 nanograms (ng) of template DNA. The size and quality of PCR products were verified by agarose gel electrophoresis. Negative controls were included in all reaction batches, and no false-positive products were observed. The number of PCR cycles was optimized at 25 to avoid overamplification, which results in artifacts. To test for amplification of pseudogene sequences, PCR reactions under the above conditions were run with 100 ng of ρo total DNA from cells devoid of mitochondrial DNA that are deficient in aerobic respiration. Cells devoid of mitochondrial DNA are shown in Schon et al. 1999 J. Med. Genet. 36:505-510, which is hereby incorporated by reference in its entirety. The primer pairs tested produced very faint bands, about 1% of the intensity seen with a control DNA template, other pairs did not amplify any products.

### Example 3

### Cloning and Sequencing of PCR Products

PCR products obtained after minigel verification (95 μl) were purified with the Qiaquick kit (Qiagen Inc.; Valencia, Calif.), mixed with a loading buffer containing 0.2% of crystal violet, and analyzed on a 1% agarose gel stained with 0.0001% crystal violet. DNA bands were excised and recovered from gel slices with the Qiaquick Gel Extraction Kit (Qiagen). DNA bands were visualized using crystal violet rather than ethidium bromide to minimize UV damage to DNA during preparation. The concentration and purity of DNA was determined spectrophotomenically at 260 nm.

Freshly purified PCR products were ligated to vector pCR 4Blunt-TOPO (Zero Blunt PCR Cloning Kit for Sequencing, Invitrogen). TOP10 Chemically Competent cells were transformed according to manufacturer's instructions, and were plated on carbenicillin LB agar plates, and plates were incubated at 30° C. for 24 hours to 30 hours. For each PCR product, 96 colonies were selected and each was placed into a well of a 96-well block (Qiagen) and was incubated with Terrific Broth (Sigma Aldrich Inc., St. Louis, Mo.) and carbenicillin, and grown at 30° C. with shaking for 30 hours to 36 hours. Plasmid DNA was purified using the Qiaquick Miniprep Kit (Qiagen) on a BioRobot 9600 (Qiagen), according to manufacturer's protocol. Concentration of DNA was adjusted to 50 ng/μl, and a seven microliter (μl) sample was used for sequencing reactions using the PCR Cycle Sequencing Kit (Amersham Biosciences; Piscataway, N.J.). Excess dyes were removed with AutoSeq plates (Amersham Biosciences), and sequencing reactions were analyzed with Applied Biosystems ABI-Prism 3700 DNA Analyzer. Plates for analysis after more than three hours were vacuum dried and re-suspended in Megabace Loading Solution (Amersham Biosciences).

### Example 4

### Sequencer Data Analysis

The raw sequencer output was analyzed with the Phred base-calling software (CodonCode; Dedham, Mass.), and data were transferred for further processing to the Sequencher sequence analysis suite (Gene Codes Corporation; Ann Arbor, Mich.). Sequences were assembled and compared to the amended Cambridge human consensus mtDNA sequence. All mutations were re-analyzed by visual inspection of the chromatograms. Only unequivocally mutated bases were included in the mutation database. Clones with more than excessive mutations or with large rearrangements were excluded from the analysis, because of the possibility of derivation from nuclear pseudogenes. Rejected clones were not preferentially observed in the fragments that having a weak ability to amplify DNA out of ρo template.

ANOVA and ANOVA on ranks were used for statistical analysis, including repeated measurements. Generalized estimating equations as implemented in SAS as PROC GENMOD were also used, controlling for the repeated measures for the multiple clones derived from each individual and for the repeated measures for the multiple genes sequenced for each clone. Thus the analysis was able to accommodate the “double clustering” of the study design.

Additional analysis of the data was performed using a genetic algorithm approach, combined with an evolutionary conservation profile (Ng and Henikoff Nucl. Acids Res. 31 (2003) 3812) and an amino acid dissimilarity index (Xia and Xie, Mol. Biol. Evol. 19 (2002) 58). The mutations were scored according to their location within genes and the type of amino acid substitutions. The modified scores were used as input for a genetic algorithm that identified areas within each gene correlating with AD status and that produced classifying functions for differentiating AD compared to control. The validity of the functions was analyzed by performing the whole computation with subsets of patients and using the resulting classifying function to classify the remaining patient (the “leave-one-out” tests).

### Example 5

### Analysis of Genes with AD Patients

Examples herein show data obtained from sequencing thousands of bases, spanning the mitochondrial genes: CO1, CO2, CO3, ATP-6 and ATP-8. Genes were analyzed and data showed that the most clearly identifiable mutations and differences between AD samples were observed in a region of the CO3 gene. No region of CO1, CO2, ATP-6 or ATP-8 was observed to have as high a degree of specificity as a region of CO3. Number of subjects having at least one CO3 mutation detected is shown in Table 2.

TABLE 2
Number of subjects with at least one mitochondrial CO3 mutation as a
function of codon region

| Codon | Control | | Control | | |
|---|---|---|---|---|---|
| number | brain | AD brain | blood | AD blood | Total |
| 1-25 | 3 | 2 | 0 | 3 | 8 |
| 26-50 | 2 | 3 | 0 | 4 | 9 |
| 51-75 | 1 | 1 | 1 | 5 | 8 |
| 76-100 | 0 | 5 | 3 | 7 | 15 |
| 101-125 | 3 | 4 | 0 | 4 | 11 |
| 126-150 | 1 | 2 | 3 | 1 | 7 |
| 151-175 | 5 | 2 | 0 | 3 | 10 |
| 176-200 | 4 | 4 | 1 | 4 | 13 |
| 201-225 | 4 | 3 | 1 | 4 | 12 |
| 226-250 | 2 | 4 | 2 | 4 | 12 |
| 251-261 | 1 | 0 | 1 | 3 | 5 |
| Total | 26 | 30 | 12 | 42 | 110 |

/

TABLE 3
Number of mitochondrial CO3 mutations in subjects as a function of
codon region

| Codon | Control | | Control | | |
|---|---|---|---|---|---|
| numbers | brain | AD brain | blood | AD blood | Total |
| 0-25 | 3 | 2 | 0 | 5 | 10 |
| 26-50 | 2 | 4 | 0 | 4 | 10 |
| 51-75 | 1 | 1 | 1 | 8 | 11 |
| 76-100 | 0 | 8 | 3 | 9 | 20 |
| 101-125 | 4 | 4 | 0 | 4 | 12 |
| 126-150 | 1 | 2 | 3 | 1 | 7 |
| 151-175 | 5 | 2 | 0 | 5 | 12 |
| 176-200 | 6 | 5 | 1 | 6 | 18 |
| 201-225 | 4 | 3 | 1 | 6 | 14 |
| 226-250 | 2 | 5 | 2 | 5 | 14 |
| 251-261 | 1 | 0 | 1 | 3 | 5 |
| Total | 29 | 36 | 12 | 56 | |

A total of 133 heteroplasmic mutations were observed in all of the subjects including AD subjects and control subjects. The nine control brain subjects had a total of 29 mutations (average of three mutations per subject); nine AD brain subjects had a total of 36 mutations (average of four mutations per subject), five control blood subjects had a total of 12 mutations (average of two mutations per subject, and 13 AD blood subjects had a total of 56 mutations (average of four mutations per subject). Thus, AD brain and blood subjects were observed to have between 33-100% more mutations that the corresponding control subjects. The greatest number of CO3 mutations were observed between codons 76-100, with 17 out of the 20 mutations occurring in AD brain and AD blood, followed by codons 176-200 (18 mutations), codons 201-225 (14 mutations), codons 226-250 (14 mutations), codons 101-125 (12 mutations, and codons 51-75 (11 mutations).

TABLE 4
Total number of mitochondrial CO3 mutations in each subject

| | | Number | | |
|---|---|---|---|---|
| | AD | of | | Codon position of mtDNA CO3 |
| Subject | status^{a} | mutations | Tissue | mutation |
| 1 | | 1 | brain | 196 |
| 2 | | 3 | brain | 158, 183, 207 |
| 3 | | 2 | brain | 115, 212 |
| 4 | | 3 | brain | 39, 62, 161 |
| 5 | | 2 | brain | 18, 207 |
| 6 | | 6 | brain | 14, 32, 107, 154, 218, 256 |
| 7 | | 3 | brain | 180, 194, 229 |
| 8 | | 4 | brain | 20, 156, 198, 200 |
| 9 | | 5 | brain | 109, 113, 148, 164, 245 |
| 10 | + | 3 | brain | 120, 201, 223 |
| 11 | + | 1 | brain | 170 |
| 12 | + | 5 | brain | 14, 38, 42, 59, 89 |
| 13 | + | 3 | brain | 41, 167, 240 |
| 14 | + | 3 | brain | 50, 85, 200 |
| 15 | + | 2 | brain | 85, 124 |
| 16 | + | 7 | brain | 77, 91, 91, 111, 198, 200, 233 |
| 17 | + | 8 | brain | 4, 91, 91, 124, 135, 179, 215, |
| | | | | 240 |
| 18 | + | 4 | brain | 139, 177, 237, 239 |
| 19 | + | 5 | blood | 80, 183, 237, 248, 254 |
| 20 | + | 2 | blood | 93, 116 |
| 21 | + | 2 | blood | 40, 212 |
| 22 | | 4 | blood | 66, 85, 144, 225 |
| 23 | + | 8 | blood | 12, 19, 77, 80, 152, 181, 192, |
| | | | | 192 |
| 24 | + | 7 | blood | 58, 59, 63, 120, 155, 161, 174 |
| 25 | | 1 | blood | 132 |
| 26 | + | 7 | blood | 46, 70, 106, 161, 176, 219, 240 |
| 27 | + | 5 | blood | 43, 59, 77, 127, 211 |
| 28 | + | 2 | blood | 75, 241 |
| 29 | + | 1 | blood | 102 |
| 30 | + | 5 | blood | 3, 93, 100, 197, 246 |
| 31 | + | 0 | blood | — |
| 32 | + | 9 | blood | 5, 24, 60, 68, 94, 210, 210, 218, |
| | | | | 260 |
| 33 | + | 3 | blood | 50, 79, 261 |
| 34 | | 1 | blood | 93 |
| 35 | | 4 | blood | 80, 191, 242, 248 |
| 36 | | 2 | blood | 139, 254 |

^{a}+ indicates that the subject was diagnosed with AD.

A large number of CO3 mutations were observed to be located between about codon position 50 to about codon position 125 and codon position 151 to about codon position 250. Analysis of the CO3 codon mutations and the amino acid sequence of CO3 (SEQ ID NO: 6) shows that codons at positions 50-125 and at positions 150-261 specify hydrophobic amino acids (i.e., alanine, isoleucine, leucine, valine, phenylalanine, tryptophan, and tyrosine are encoded at codons 52, 55, 57-58, 67, 75, 79, 81, 84-87, 91-95, 97-82, 106-107, 112, 116, 121, 124, 127, 129, 151, 160, 162-165, 167-169, 171-173, 175-176, 178, 181-182, 186, 192-193, 197-200, 203, 206, 208-210, 214-215, 217, 219-220, 223, 225, 227, 233, 235, 237-242, 244-245, 247-254, and 256-258). Without being limited by any particular theory or mechanism of action, it is here envisioned that the heteroplasmic CO3 gene mutations observed in brain and bloods samples from AD subjects are correlated with mutations that result in substitution of hydrophilic (i.e., less hydrophobic) amino acids such as cysteine, asparagines, glutamine, methionine, serine, aspartic acid, threonine, and glutamic acid. Thus, by reducing the relative hydrophobicity of the CO3 enzyme, the CO3 gene mutations in AD patients affect the folding, binding, and stability of the enzyme, thereby negatively affecting the activity of the CO3 enzyme and resulting in AD in the subjects. These mutations are associated with a reduction in cytochrome c oxidase activity and AD in subjects.

### Example 6

### Mitochondrial CO3 Mutations Diagnose AD

To confirm correlation of presence of amino acid changing mutations in codon regions of CO3 genes with ADin subject, a large number of samples from AD patients and blood samples from normal control subjects are analyzed for amino acid changing CO3 gene mutations. Cells are obtained and DNA extracted and subjected to PCR amplification (100-300 clones) to identify mtDNA CO3 mutations. The data demonstrate presence of amino acid changing heteroplasmic mutations in CO3 gene and are correlated with the prognosis or diagnosis of AD. Analyses are performed using primers to amplify CO3 mutations. Comparison of the mutant proteins is performed using three-dimensional protein structure analysis. Analysis of the proteins compared to control proteins shows that the mutated proteins have altered protein folding, binding kinectics, and other protein characteristics, including potential differences in the open reading frame length, molecular weight, isoelectric point (pI), and hydrophobicity and hydrophilicity. The mutated proteins are tested for cytochrome c oxidase activity in vivo and in pure form. It is expected that a portion of these proteins have lower enzyme activity (e.g., CO activity) than control proteins from normal subjects that do not have AD.

Examples include obtaining blind samples from normal subjects and AD subjects, and analyzing these samples for CO3 gene mutations. Using methods described herein, these samples are decoded and identified and those as having AD at the time of the analysis are correlated with presence of CO3 gene mutations. Data analysis includes determining whether subjects were previously identified as being at risk for having AD, or whether those subjects later developed AD. Data show that methods herein are effective for diagnosing AD in a human subject, the methods include determining the presence or absence in the subject of a heteroplasmic, amino acid changing mutation in a region of the mitochondrial cytochrome c oxidase subunit III (CO3) gene.

1-37. (canceled)

38. A method of diagnosing a presence or a risk for a human subject to develop Alzheimer's disease, comprising determining a presence in the subject of a low abundance heteroplasmic amino acid changing mutation in a region of a gene encoding a mitochondrial cytochrome c oxidase subunit III (CO3) gene, wherein the region comprises codons 45-250, wherein the presence of the low abundance heteroplasmic amino acid changing mutation encoding one or more of codons 45-250 of the gene is an indication that the human subject has the risk to develop Alzheimer's Disease.

39. The method according to any of claim 38, wherein the mutation comprises at least one nucleotide change selected from a substitution.

40. The method according to claim 38, wherein the sample comprises at least one of: a cell, a fluid, and a tissue.

41. The method according to claim 40, wherein the fluid is at least one selected from the group consisting of: blood, serum, plasma, mucus, saliva, cerebrospinal fluid, semen, tears, and urine.

42. The method according to claim 40, wherein the cell or the tissue is selected from at least one selected from the group consisting of: vascular, epithelial, endothelial, dermal, dental, connective, muscular, neuronal, facial, cranial, soft tissue, cartilage, collagen, brain, bone, and bone marrow.

43. The method according to claim 38, wherein the mutation encodes an amino acid change in at least one of codon 45 to codon 60, codon 64 to codon 103, 75 to codon 100, 120 to codon 145, and 225 to codon 250.

44. The method according to claim 38, wherein further comprising obtaining nucleic acid from the sample.

45. The method according to claim 44 further comprising amplifying at least one nucleotide sequence, wherein amplifying comprises hybridizing to at least one primer or probe specific for a portion of the extracted nucleic acid.

46. The method according to claim 45, wherein amplifying comprises at least one method selected from the group of: polymerase chain reaction (RT-PCR), branched DNA signal amplification, ligase chain reaction, isothermal nucleic acid sequence based amplification (NASBA), Q-beta replication, transcription-based amplification, an amplifiable RNA reporter, boomerang DNA amplification, strand displacement activation, cycling probe technology, and a sequence replication assay.

47. The method according to claim 45, wherein the primer or the probe comprises at least one nucleotide sequence of CO3 gene.

48. The method according to claim 45, wherein the primer or the probe is attached to a matrix or scaffold.

49. The method according to claim 48, wherein the matrix or scaffold comprises at least one selected from the group of: a metal, a plastic, and a polymer.

50. The method according to claim 48, wherein the matrix or scaffold comprises an organic material or inorganic material.

51. The method according to claim 45 further comprising ligating at least one nucleotide sequence to a vector.

52. The method according to claim 45, wherein amplifying comprises constructing a plurality of clones from the extracted nucleic acid.

53. The method according to claim 52, wherein the plurality of clones comprises at least about 50 clones, at least about 100 clones, at least about 150 clones, at least about 200 clones, or at least about 300 clones.

54. The method according to claim 45 further comprising detecting the presence of the heteroplasmic amino acid changing mutation in the mitochondrial nucleotide sequence.

55. The method according to claim 54, wherein detecting comprises at least one selected from the group of: electrophoresis, an array comparative genomic hybridization (CGH), an immunoassay, an immunological detection, fluorescence, chemiluminescence, and chromatography.

56. The method according to claim 55, wherein detecting further comprises analyzing using an analytical device.

57. The method according to claim 56, wherein the analytical device comprises a computer.

58. The method according to claim 56, wherein the analytical device comprises a sequence analyzer.

59. The method according to claim 38, wherein determining further comprises identifying the presence of the heteroplasmic amino acid changing mutation in a sample from at least one family relative selected from: a sibling, a cousin, a parent, a grandparent, an aunt, an uncle, and a child.

60. A method for treatment, diagnosis, prognosis, genetic counseling, or psychosocial management related to Alzheimer's disease (AD) in a subject, the method comprising:
obtaining a plurality of nucleotide sequences from a sample from the subject, the nucleotide sequences encoding at least one mitochondrial cytochrome c oxidase (CO) subunit 3 gene; and,
comparing the sequences of the mitochondrial gene in the sample from the subject to a control mitochondrial sequence and determining an absence or a presence in the sample of a low abundance heteroplasmic amino acid changing mutation in a region of the gene encoding the CO3 gene, wherein the region comprises codons 45-250, wherein the presence of the heteroplasmic amino acid changing mutation encoding one or more of codons 45-250 of the gene is an indication that the subject has a risk of developing AD, thereby achieving the treatment, the diagnosis, the prognosis, the genetic counseling, or the psychosocial management related to AD.

61. The method according to claim 60, wherein the AD is familial AD.

62. The method according to claim 60, wherein the AD is sporadic AD.

63. The method according to claim 60, wherein the control mitochondrial sequence comprises at least one nucleotide sequence selected from the group of: SEQ ID NO: 1, SEQ ID NO: 3, SEQ ID NO: 5, SEQ ID NO: 7, and SEQ ID NO: 9.

64. The method according to claim 60, wherein the control mitochondrial sequence comprises an amino acid sequence selected from the group of: SEQ ID NO: 2, SEQ ID NO: 4, SEQ ID NO: 6, SEQ ID NO: 8, and SEQ ID NO: 10.

65. The method according to claim 60, wherein prior to comparing, the method comprises obtaining nucleic acid from the sample.

66. The method according to claim 65, wherein obtaining comprises amplifying the nucleic acid and constructing a plurality of clones comprising at least about 50 clones, at least about 100 clones, at least about 150 clones, at least about 200 clones, or at least about 300 clones.

67. The method according to claim 65, wherein the mutation comprises at least one nucleotide change selected from a substitution.

68. The method according to claim 65, wherein the sample comprises at least one of: a cell, a fluid, and a tissue.

69. The method according to claim 68, wherein the fluid is at least one selected from the group consisting of: blood, serum, plasma, mucus, saliva, cerebrospinal fluid, semen, tears, and urine.

70. The method according to claim 68, wherein the cell or the tissue is selected from at least one selected from the group consisting of: vascular, epithelial, endothelial, dermal, dental, connective, muscular, neuronal, facial, cranial, soft tissue, cartilage and collagen, brain, bone, and bone marrow.

71. A method of treating a human subject having Alzheimer's disease (AD) or at risk for AD, the method comprising:
identifying by nucleotide sequencing at least one mismatch in a mitochondrial cytochrome c oxidase subunit III (CO3) gene between a human control not having AD and the subject, wherein the mismatch comprises at least one low abundance heteroplasmic amino acid changing mutation in a nucleotide sequence in a region of the gene, wherein the region comprises codons 45-250, thereby determining that mitochondria from the subject have the mutation;
constructing a negative modulator that down regulates expression or function of the specific nucleotide sequence encoding the mutation; and,
contacting a cell or a tissue of the subject with the modulator, thereby down regulating expression of the mutation and treating the subject having AD or at the risk for AD.

72. The method according to claim 71, wherein the negative modulator comprises a small interfering RNA (siRNA) that specifically targets the nucleotide sequence encoding the mismatch or that encodes an agent that binds to the mismatch.


SEQUENCE LISTING (22 sequences)

SEQ ID NO: 1 - DNA, 1542 bp, Homo sapiens
atgttcgccgaccgttgactattctctacaaaccacaaagacattggaacactatacctattattcggcgcatgagctggagtcctaggcacagctctaagcctccttattcgagccgagctgggccagccaggcaaccttctaggtaacgaccacatctacaacgttatcgtcacagcccatgcatttgtaataatcttcttcatagtaatacccatcataatcggaggctttggcaactgactagttcccctaataatcggtgcccccgatatggcgtttccccgcataaacaacataagcttctgactcttacctccctctctcctactcctgctcgcatctgctatagtggaggccggagcaggaacaggttgaacagtctaccctcccttagcagggaactactcccaccctggagcctccgtagacctaaccatcttctccttacacctagcaggtgtctcctctatcttaggggccatcaatttcatcacaacaattatcaatataaaaccccctgccataacccaataccaaacgcccctcttcgtctgatccgtcctaatcacagcagtcctacttctcctatctctcccagtcctagctgctggcatcactatactactaacagaccgcaacctcaacaccaccttcttcgaccccgccggaggaggagaccccattctataccaacacctattctgatttttcggtcaccctgaagtttatattcttatcctaccaggcttcggaataatctcccatattgtaacttactactccggaaaaaaagaaccatttggatacataggtatggtctgagctatgatatcaattggcttcctagggtttatcgtgtgagcacaccatatatttacagtaggaatagacgtagacacacgagcatatttcacctccgctaccataatcatcgctatccccaccggcgtcaaagtatttagctgactcgccacactccacggaagcaatatgaaatgatctgctgcagtgctctgagccctaggattcatctttcttttcaccgtaggtggcctgactggcattgtattagcaaactcatcactagacatcgtactacacgacacgtactacgttgtagcccacttccactatgtcctatcaataggagctgtatttgccatcataggaggcttcattcactgatttcccctattctcaggctacaccctagaccaaacctacgccaaaatccatttcactatcatattcatcggcgtaaatctaactttcttcccacaacactttctcggcctatccggaatgccccgacgttactcggactaccccgatgcatacaccacatgaaacatcctatcatctgtaggctcattcatttctctaacagcagtaatattaataattttcatgatttgagaagccttcgcttcgaagcgaaaagtcctaatagtagaagaaccctccataaacctggagtgactatatggatgccccccaccctaccacacattcgaagaacccgtatacataaaatctaga

SEQ ID NO: 2 - protein, 513 aa, Homo sapiens
MFADRWLFSTNHKDIGTLYLLFGAWAGVLGTALSLLIRAELGQPGNLLGNDHIYNVIVTAHAFVMIFFMVMPIMIGGFGNWLVPLMIGAPDMAFPRMNNMSFWLLPPSLLLLLASAMVEAGAGTGWTVYPPLAGNYSHPGASVDLTIFSLHLAGVSSILGAINFITTIINMKPPAMTQYQTPLFVWSVLITAVLLLLSLPVLAAGITMLLTDRNLNTTFFDPAGGGDPILYQHLFWFFGHPEVYILILPGFGMISHIVTYYSGKKEPFGYMGMVWAMMSIGFLGFIVWAHHMFTVGMDVDTRAYFTSATMIIAIPTGVKVFSWLATLHGSNMKWSAAVLWALGFIFLFTVGGLTGIVLANSSLDIVLHDTYYVVAHFHYVLSMGAVFAIMGGFIHWFPLFSGYTLDQTYAKIHFTIMFIGVNLTFFPQHFLGLSGMPRRYSDYPDAYTTWNILSSVGSFISLTAVMLMIFMIWEAFASKRKVLMVEEPSMNLEWLYGCPPPYHTFEEPVYMKS

SEQ ID NO: 3 - DNA, 684 bp, Homo sapiens
atggcacatgcagcgcaagtaggtctacaagacgctacttcccctatcatagaagagcttatcacctttcatgatcacgccctcataatcattttccttatctgcttcctagtcctgtatgcccttttcctaacactcacaacaaaactaactaatactaacatctcagacgctcaggaaatagaaaccgtctgaactatcctgcccgccatcatcctagtcctcatcgccctcccatccctacgcatcctttacataacagacgaggtcaacgatccctcccttaccatcaaatcaattggccaccaatggtactgaacctacgagtacaccgactacggcggactaatcttcaactcctacatacttcccccattattcctagaaccaggcgacctgcgactccttgacgttgacaatcgagtagtactcccgattgaagcccccattcgtataataattacatcacaagacgtcttgcactcatgagctgtccccacattaggcttaaaaacagatgcaattcccggacgtctaaaccaaaccactttcaccgctacacgaccgggggtatactacggtcaatgctctgaaatctgtggagcaaaccacagtttcatgcccatcgtcctagaattaattcccctaaaaatctttgaaatagggcccgtatttaccctatag

SEQ ID NO: 4 - protein, 227 aa, Homo sapiens
MAHAAQVGLQDATSPIMEELITFHDHALMIIFLICFLVLYALFLTLTTKLTNTNISDAQEMETVWTILPAIILVLIALPSLRILYMTDEVNDPSLTIKSIGHQWYWTYEYTDYGGLIFNSYMLPPLFLEPGDLRLLDVDNRVVLPIEAPIRMMITSQDVLHSWAVPTLGLKTDAIPGRLNQTTFTATRPGVYYGQCSEICGANHSFMPIVLELIPLKIFEMGPVFTL

SEQ ID NO: 5 - DNA, 784 bp, Homo sapiens
atgacccaccaatcacatgcctatcatatagtaaaacccagcccatgacccctaacaggggccctctcagccctcctaatgacctccggcctagccatgtgatttcacttccactccataacgctcctcatactaggcctactaaccaacacactaaccatataccaatgatggcgcgatgtaacacgagaaagcacataccaaggccaccacacaccacctgtccaaaaaggccttcgatacgggataatcctatttattacctcagaagtttttttcttcgcaggatttttctgagccttttaccactccagcctagcccctaccccccaattaggagggcactggcccccaacaggcatcaccccgctaaatcccctagaagtcccactcctaaacacatccgtattactcgcatcaggagtatcaatcacctgagctcaccatagtctaatagaaaacaaccgaaaccaaataattcaagcactgcttattacaattttactgggtctctattttaccctcctacaagcctcagagtacttcgagtctcccttcaccatttccgacggcatctacggctcaacattttttgtagccacaggcttccacggacttcacgtcattattggctcaactttcctcactatctgcttcatccgccaactaatatttcactttacatccaaacatcactttggcttcgaagccgccgcctgatactggcattttgtagatgtggtttgactatttctgtatgtctccatctattgatgagggtctt

SEQ ID NO: 6 - protein, 261 aa, Homo sapiens
MTHQSHAYHMVKPSPWPLTGALSALLMTSGLAMWFHFHSMTLLMLGLLTNTLTMYQWWRDVTRESTYQGHHTPPVQKGLRYGMILFITSEVFFFAGFFWAFYHSSLAPTPQLGGHWPPTGITPLNPLEVPLLNTSVLLASGVSITWAHHSLMENNRNQMIQALLITILLGLYFTLLQASEYFESPFTISDGIYGSTFFVATGFHGLHVIIGSTFLTICFIRQLMFHFTSKHHFGFEAAAWYWHFVDVVWLFLYVSIYWWGS

SEQ ID NO: 7 - DNA, 681 bp, Homo sapiens
atgaacgaaaatctgttcgcttcattcattgcccccacaatcctaggcctacccgccgcagtactgatcattctatttccccctctattgatccccacctccaaatatctcatcaacaaccgactaatcaccacccaacaatgactaatcaaactaacctcaaaacaaatgataaccatacacaacactaaaggacgaacctgatctcttatactagtatccttaatcatttttattgccacaactaacctcctcggactcctgcctcactcatttacaccaaccacccaactatctataaacctagccatggccatccccttatgagcgggcacagtgattataggctttcgctctaagattaaaaatgccctagcccacttcttaccacaaggcacacctacaccccttatccccatactagttattatcgaaaccatcagcctactcattcaaccaatagccctggccgtacgcctaaccgctaacattactgcaggccacctactcatgcacctaattggaagcgccaccctagcaatatcaaccattaaccttccctctacacttatcatcttcacaattctaattctactgactatcctagaaatcgctgtcgccttaatccaagcctacgttttcacacttctagtaagcctctacctgcacgacaacacataa

SEQ ID NO: 8 - protein, 226 aa, Homo sapiens
MNENLFASFIAPTILGLPAAVLIILFPPLLIPTSKYLINNRLITTQQWLIKLTSKQMMTMHNTKGRTWSLMLVSLIIFIATTNLLGLLPHSFTPTTQLSMNLAMAIPLWAGTVIMGFRSKIKNALAHFLPQGTPTPLIPMLVIIETISLLIQPMALAVRLTANITAGHLLMHLIGSATLAMSTINLPSTLIIFTILILLTILEIAVALIQAYVFTLLVSLYLHDNT

SEQ ID NO: 9 - DNA, 204 bp, Homo sapiens
atgccccaactaaatactaccgtatggcccaccataattgcccccatacttcttacactattcctcatcactcagctaaaaatattaaatacaaattaccatctacccccctcaccaaagcccataaaaataaaaaactatagtaaaccctgagaaccaaagtgaacgaaaatctgttcgcttcattcattgcccccacaatcc

SEQ ID NO: 10 - protein, 68 aa, Homo sapiens
MPQLNTTVWPTMITPMLLTLFLITQLKMLNTNYHLPPSPKPMKMKNYNKPWEPKWTKICSLHSLPPQS

SEQ ID NO: 11 - DNA, 24 bp, Artificial Sequence
  The sequence was designed and synthesized
agcaccctaatcaactggcttcaa

SEQ ID NO: 12 - DNA, 21 bp, Artificial Sequence
  The sequence was designed and synthesized
cttcgcaggcggcaaagacta

SEQ ID NO: 13 - DNA, 24 bp, Artificial Sequence
  The sequence was designed and synthesized
agcaccctaatcaactggcttcaa

SEQ ID NO: 14 - DNA, 21 bp, Artificial Sequence
  The sequence was designed and synthesized
cttcgcaggcggcaaagacta

SEQ ID NO: 15 - DNA, 24 bp, Artificial Sequence
  The sequence was designed and synthesized
agcaccctaatcaactggcttcaa

SEQ ID NO: 16 - DNA, 21 bp, Artificial Sequence
  The sequence was designed and synthesized
cttcgcaggcggcaaagacta

SEQ ID NO: 17 - DNA, 20 bp, Artificial Sequence
  The sequence was designed and synthesized
gccctagcccacttcttacc

SEQ ID NO: 18 - DNA, 20 bp, Artificial Sequence
  The sequence was designed and synthesized
ttaaggcgacagcgatttct

SEQ ID NO: 19 - DNA, 20 bp, Artificial Sequence
  The sequence was designed and synthesized
ccgactaatcaccacccaac

SEQ ID NO: 20 - DNA, 20 bp, Artificial Sequence
  The sequence was designed and synthesized
gatggccatggctaggttta

SEQ ID NO: 21 - DNA, 20 bp, Artificial Sequence
  The sequence was designed and synthesized
gtatggcccaccataattgc

SEQ ID NO: 22 - DNA, 20 bp, Artificial Sequence
  The sequence was designed and synthesized
gcaatgaatgaagcgaacag